(19) United States
(12) Patent Application Publication
(10) Pub. No.: US 2002/0192271 A1
(43) Pub. Date: Dec. 19, 2002

(54) Method for causing local hemostasis and hemostatic composition for local hemostasis

(76) Inventors: Ulla Karin Elisabeth Hedner, Malmo (SE); Erik Hogsbro Ostergaard, Varlose (DK); Martin William Edwards, Woodinville, WA (US)

Correspondence Address: Reza Green, Esq., Novo Nordisk of North America, Inc., Suite 6400, 405 Lexington Avenue, New York, NY, 10174-6401, US

(21) Appl. No.: 10/224,717

(22) Filed: Aug. 13, 2002

Related U.S. Application Data
(63) Continuation of application No. 09/712,760, filed on Nov. 14, 2000, now abandoned.
Application No. 09/712,760 is a continuation of application No. 09/197,068, filed on Nov. 20, 1998, now abandoned.
Application No. 09/197,068 is a continuation of application No. 07/775,664, filed on Oct. 11, 1991, now abandoned.
Application No. 07/775,664 is a continuation-in-part of application No. 07/666,423, filed on Mar. 8, 1991, now Pat. No. 5,180,583.
Application No. 07/666,423 is a continuation of application No. 07/368,967, filed on Jun. 19, 1989, now abandoned.
Application No. 07/368,967 is a continuation of application No. 06/933,408, filed on Nov. 20, 1986, now abandoned.

(30) Foreign Application Priority Data
Nov. 26, 1985 (DK) DK5446/85
Sep. 26, 1986 (DK) DK4592/86

(51) Int. Cl. A61K 38/36; A61L 15/00

(52) U.S. Cl. 424/445; 514/12; 442/123

(57) ABSTRACT

Method for arresting local bleedings by topical use of FVIIa and a hemostatic composition containing FVIIa.

### RELATED APPLICATIONS

The present invention is a continuation-in-part of U.S. Ser. No. 07/666,423, filed Mar. 8, 1991, which is a continuation of U.S. Ser. No. 07/368,967, filed Jun. 19, 1989, which is a continuation of U.S. Ser. No. 06/933,408, filed Nov. 20, 1986 all of which are incorporated herein by reference in their entirety.

### FIELD OF INVENTION

The present invention relates to a method for arresting local bleedings by topical use of FVIIa and a hemostatic composition containing FVIIa.

### BACKGROUND OF THE INVENTION

When blood vessels are injured by physical traumas including surgical interventions bleeding will occur. If bleedings are left alone they will eventually be arrested by a normally occurring physiological process characterized by a chain of events involving the combined activity of vascular, platelet, and plasma factors, leading to the formation of a blood clot. This process is referred to as physiological hemostasis (blood coagulation), which is described in details below. In the case of a minor superficial bleeding this physiological hemostasis is adequate for the arrest.

There are two separate systems which can promote blood coagulation. These systems are referred to as the intrinsic and the extrinsic coagulation pathways.

In the intrinsic pathway, only blood clotting factors present in plasma are utilized. An intermediate event in the intrinsic pathway is the activation of Factor IX to Factor IXa, a reaction catalyzed by Factor XIa and calcium ions. Factor IXa then participates in the activation of Factor X to Factor Xa in the presence of Factor VIIIa, phospholipid and calcium ions.

The extrinsic pathway involves plasma factors as well as components present in tissue extracts. Factor VII, a proenzyme present in plasma, participates also in the extrinsic pathway of blood coagulation by converting (upon its activation to VIIa) Factor X to Xa in the presence of tissue factor and calcium ions.

Factor Xa in turn then converts prothrombin to thrombin in the presence of Factor Va, calcium ions and phospholipid. Finally, thrombin converts the plasma fibrinogen into fibrin, which in the presence of Factor XIIIa and calcium ions is cross-linked and thus forming the blood clot.

Blood factors such as Factor VIII:C (see U.S. Pat. Nos. 4,831,119; 4,868,112; 4,886,876; 4,657,894; Re. 32,011 and 4,649,132) and Factor VIIa (see U.S. Pat. Nos. 4,784,950; 4,382,083; 4,479,938 and 4,357,321 all of which are incorporated herein by reference in their entirety) purified from natural sources or made via recombinant techniques have been used for treating patients, such as hemophiliacs, having blood-clotting deficiencies or inhibitors to blood-clotting factors. These blood-clotting factors have been delivered to the patient needing treatment as an aqueous solution by infusion or bolus injection depending on the blood factor to be delivered and the condition of the patient. Cessation of the bleeding is expected to occur typically between 15 minutes to 3 hours or more after the delivery of the blood-clotting factor.

However, faster arresting of the bleeding is necessary in the case of severe bleedings emerging from more extensive injuries involving larger arteries or when seeping bleedings occur from larger mucosal surfaces or on cavities without drainage. If the bleeding continues in even a shorter period it may result in extensive losses of blood which may have an adverse effect on the normal function of the body. Also, in the case of bleeding occurring in osseous non-expandable cavities, the accumulation of extravasated blood may cause local damages of soft tissues due to increased pressure. The usual treatment of such conditions involve the adaption of surgical and/or medical hemostatic measures.

Surgical arrest of bleeding comprises ligation or suture of disrupted blood vessels, plugging by using tampons in cavities, coagulating tissue surfaces including their exposed disrupted blood vessels by heated instruments or by the application of cauterizing agents or heated air.

Surgical hemostasis may also be aided by the application at the injured site of appropriately sized blocks, plates, or films of biologically absorbable hemostatic sponges.

Pharmaceutical preparations containing bovine thrombin or other blood clotting factors such as Factor VIII, Factor XIII or calcium ions are currently used in some places as hemostatic adjuncts in surgery, said adjuncts being administered e.g. by spraying a suitable solution thereof onto the site of bleeding such as in U.S. Pat. No. 4,298,598. Also textile materials such as gauze or cotton wool fabrics or biologically absorbable sponges, which prior to the application have been soaked in a solution of one or more of said hemostatic compounds, are used such as in U.S. Pat. No. 4,363,319.

U.S. Pat. No. 2,558,395 discloses a ready-to-use undenatured gelatine hemostatic sponge containing thrombin. U.S. Pat. No. 4,265,233 discloses wound healing material comprising a structure made from compounds such as gelatine, collagen, polyglycolic and polylactic acid to which FXIII has been fixed by covalent binding. EP 277096 A discloses a wound dressing comprising a stable thrombin composition and a substrate such as hemostatic, porous sponges of collagen or denatured gelatine and WO90/13320 discloses a porous sponge containing a hemostatically effective amount of thrombin, and hemostatically effective amounts of one or more blood coagulation factors other than thrombin. U.S. Pat. Nos. 4,563,387 and 4,642,111 relate to, respectively, a method and device for treating cancer and which disclose an anti-cancer drug and a blood coagulation factor being fixed to a structure, such as a polymer, capable of being delivered by injection to the site of bleeding directly caused by the cancer treatment.

Japanese published patent application No. 59-116213 discloses an aerosol containing FXIII and thrombin and Japanese published patent application No. 02-167234 discloses adhesive for living tissues containing fibrinogen, prothrombin, FVII, FIX, FX, FXIII, antithrombin, protease inhibitor and calcium ions.

In the recent years increasing concern has however arisen regarding the safe use of bovine derived products e.g. thrombin or prothrombin in pharmaceutical products for human use. Several reports describe the possible risk of transmitting an infectious agent causing Bovine Spongiform Encephalopathy (BSE) in cattle into humans, where the virus-like agent may be the reason for one or more well known diseases characterized by degenerative encephalopathy e.g. Creutzfeldt-Jacob disease and Kuru. Furthermore, clinical investigators have observed that the topical use of bovine thrombin in humans may cause the development of antibodies cross-reacting to human thrombin and causing bleeding problems.

It is, therefore, an object of the present invention to provide a safe and effective means to topically arrest bleedings at the site of an injury.

### SUMMARY OF THE INVENTION

The present invention is based on the surprising recognition that FVIIa is capable of momentarily arrest of bleedings when applied topically to the site of injury without the presence of thrombin or other coagulation factors.

In its broadest aspect the present invention is related to the method for arresting bleedings wherein a hemostatically effective amount of FVIIa is applied topically to the site of injury without addition of thrombin.

More specifically, this invention provides a method for inducing hemostasis at a bleeding wound comprising providing topically to the site of the bleeding wound a hemostatically effective amount of FVIIa, which is uncontaminated by other blood clotting factors and which has sufficient activity alone to produce a hemostatic effect, together with a biologically compatible carrier. The inventors hereof have furthermore ascertained that optimal effect is achieved when FVIIa is in association, together with or incorporated into a biologically acceptable carrier (which, as used herein, is intended to include a composition or material) capable of preventing FVIIa from being washed away from the site of injury.

In the present method FVIIa is incorporated into a biologically compatible carrier which does not contain thrombin and is unaccompanied or uncontaminated by any other blood clotting factors.

The present invention is also related to a hemostatic composition comprising a hemostatically effective amount of FVIIa incorporated into biologically compatible carrier said composition containing no thrombin. More specifically, this invention provides a hemostatic composition for inducing hemostasis at a bleeding wound comprising a hemostatically effective amount of FVIIa which is uncontaminated or unaccompanied by other blood clotting factors and which has sufficient activity alone to produce a hemostatic effect, together with a biologically compatible carrier which permits said factor VIIa to remain affixed to, in association with or contacting said wound site.

Definitions

Prior to setting forth the invention, it may be helpful to an understanding thereof to set forth definition of certain terms to be used hereinafter.

Hemostat or Hemostatic Agent: An agent that arrests hemorrhage.

Hemostatic Composition: A composition that contains a Hemostat or Hemostatic Agent.

Blood clot: The final outcome of the blood coagulation cascade, formed by conversion of soluble plasma fibrinogen into insoluble fibrin, which physically stops the bleeding. The blood clot covers the surface, keeps the wound edges together and forms the matrix for the following cell proliferation and wound healing.

Blood clotting factors: Plasma proteins which participate in the blood coagulation cascade.

Activated blood clotting factors: Blood clotting factors converted to active enzymes by the action of an activator, often itself being an activated blood clotting factor. They are generally designated by the addition of a lower case postscript “a” (e.g. Factor VIIa).

Proenzymes: An enzyme precursor that in general has reduced or no activity as compared to the mature enzyme.

Biologically absorbable: Material which can be degraded in the body to smaller molecules having a size which allows transport into the blood stream and gradual removal from the site of application.

Sponge: A porous structure being reticulate and having an inner surface considerably larger than the outer surface. The porous structure will contain hollow spaces within the reticulate structure and can absorb many times its own weight in liquids.

Covalent binding: A bond between two atoms in which both of the atoms concerned contribute the electron or electrons.

Dressing: Material applied to a wound and fastened in place to provide protection and to promote healing.

Topical: Local.

Biologically Compatible: The ability to be accepted in the body and remain functional for a period without rejection.

Gel: A colloidal system comprising a solid and a liquid phase which exists as a solid or semisolid mass.

Paste: An ointment-like preparation of one or more substances in a hydrogel or fatty base. It is less greasy and better absorbed than an ointment.

Granule: A minute particle or mass.

Film: Any thin covering, coating, or layer.

Plaster: A substance intended for external application, made of such material and of such consistency as to adhere to the skin.

Bandage: A strip of gauze, muslin, flannel, or other material used to hold dressings in place, or to check hemorrhage.

### DETAILED DESCRIPTION

FVIIa is to be used in a hemostatically effective amount. By hemostatically effective amount is meant an amount which will preferably cause arrest of the bleeding if kept in association with or contacting the site of the injury for a sufficient amount of time, preferably from about 60 seconds in patients not having an impaired hemostatic mechanism to less than about 10 minutes in patients having an impaired hemostatic mechanism. FVIIa should be used in an amount ranging from about 0.2 to about 2.0 mg, preferably from about 0.5 to about 1.5 mg and more preferably from about 0.9 to about 1.1 mg per application.

FVIIa may be derived from plasma as described in EP 0082182B or by recombinant DNA-technology as described in EP 0200421A. Human purified factor VIIa is preferably made by the methods described by Broze and Majerus, J. Bio. Chem. 255, 4: 1242-1247, 1980, and Hedner and Kisiel, J. Clin. Invest. 71: 1836-1841, 1983. These methods yield factor VII without detectable amounts of other blood coagulation factors.

An even further purified factor VII preparation may be obtained by including an additional gel filtration as the final purification step. Factor VII is then converted into activated factor VIIa by known means, e.g. by several different plasma proteins, such as factor XIIA, IXA OR XA. Alternatively, as described by Bjoern et al., (“Activation of Coagulation Factor VII to VIIa”, Research Disclosure 269:564-565, 1986) factor VII may be activated by passing it through an ion-exchange chromatography column, such as MonoQ (Pharmacia Fine Chemicals, Uppsala, Sweden) or the like.

It will be appreciated by those skilled in the art that a suitable factor VIIa for use in the present invention may also be produced by recombinant DNA technology, e.g., by insertion of the cDNA or gene encoding factor VII (Hagen et al., Proc. Natl. Acad. Sci. USA 83: 2412-2416, 1986) in a suitable vector, transforming of suitable cell lines with the vector and growing the transformed cells in an appropriate medium whereupon the expressed product is isolated and activated into factor VIIa. Production of FVII by recombinant DNA technology is also described in U.S. Pat. No. 4,784,950 which is incorporated herein by reference in its entirety. Factor VIIa produced by recombinant DNA technology may be authentic factor VIIa or a more or less modified factor VIIa, provided that such modified factor VIIa has substantially the same biological activity for blood coagulation as authentic factor VIIa. Such modified factor VIIa may be prepared by modifying the DNA sequence encoding factor VII either by altering the amino acid codons or by removal of some of the amino acid codons in the natural gene by known means, e.g., by site-specific mutagenesis.

It is evident that the practice of the methods described herein is independent of how the factor VIIa is derived and, therefore, the present invention is contemplated to cover the use of any factor VIIa preparation suitable for use herein.

The carrier material may be a gel, a paste, a solid or other suitable biologically compatible/acceptable carrier for topical application of pharmaceuticals or other biologically active compositions.

The viscosity of the gel or paste will preferably be from about 200 cps to about 30,000 cps.

The biologically compatible carrier will typically be made of natural macromolecules such as gelatine, collagen, alginic acid, cellulose, chitin, fibrinogen, fibrin, fibrin split products, fibronectin, fibronectin fragments, globulin, myoglobulin, casein, keratin, albumin, polysaccharides e.g. dextrans, glycosaminglycans, agar, pectin, starch or from chemical modified natural molecules such as denatured gelatine, alginicacid-alginates e.g. calcium alginate, oxidized cellulose, substituted cellulose ethers e.g. glycol cellulose, methyl cellulose, ethyl cellulose, hydroxymethyl cellulose, hydroxyethyl cellulose, hydroxypropylmethyl cellulose, substituted cellulose esters e.g. acetylated cellulose, substituted cellulose ether-esters e.g. acetylated ethyl cellulose, chithosan or from synthetic polymers such as vinyl polymers, e.g. polyacrylic acid, polymethacrylic acid, polyvinyl pyrrolidone and polyvinyl alcohol, polyglycolic acid, polylactic acid, polydextroses or copolymers such as polyoxyethylene-polyoxypropylene copolymers or from natural fibers, synthetic fibers or mixtures of any of the above materials/compounds.

A solid biologically compatible carrier will preferably be a granule, powder, sponge, film, plaster, surgical dressing or a bandage.

Solid biologically compatible carriers will typically be selected from those already used as hemostats such as modified cellulose, collagen, gelatine, alginate or synthetic polymers.

The biologically compatible carrier may furthermore contain a fibrinolysis inhibitor, such as aprotinin, epsilon-aminocaproic acid or tranexamic acid. It may also contain a stabilizer, such as naturally occurring amino acids, mono- or disaccharides, polyglycols, glycerol, proteins or a metal salt, such as calcium salts, and mixtures thereof. Also buffering salts may be added, such as alkaline metal acetates, alkaline metal carbonates or hydrogen carbonates, alkaline metal citrates, alkaline metal phosphates or hydrogen phosphates, alkaline metal succinates, imidazole, TRIS, and zwitteranionic buffering systems, and mixtures thereof. Furthermore, antimicrobial or bacteriostatic agents, such as antibiotics, sulphonamides, antimycotic agents, antiviral compounds, and preservatives may be added.

The present method and hemostatic composition will be useful for enhancing the arrest of bleedings in several instances of surgical interventions or other injuries such as in the accidental injury of the skin and/or adjacent tissues or of larger abdominal organs (liver, spleen, or intestines); in lung surgery; in neurosurgery to prevent pressure damages of the cerebral or nerve tissues; in orthopedic surgery during which extensive hemorrhages frequently occur which are difficult to arrest by other means; in vascular surgery to arrest seepage of blood from the sites of suturing; in oral or dental surgery such as extraction of teeth; and in nose-bleeding (epistaxis).

In a ready-to-use product incorporation of FVIIa into the carrier material may be done by various known methods, such as co-precipitation, swelling, dispersion, mixing, soaking, spraying, embedding, injection or a combination thereof.

If the carrier is a gel or a paste, FVIIa is preferably incorporated into the carrier material under aseptical conditions. This may be carried out by adding a suitable solution of FVIIa to the carrier material which is then stirred gently by suitable means to obtain a uniform distribution of FVIIa within the gel or paste. The FVIIa loaded carrier material is then transferred to a suitable package form e.g. a tube, a plastic container or a syringe. Terminal sterilization may be carried out by means of, for instance, heat or ionizing irradiation.

If the carrier is solid it may be loaded with FVIIa by placing the material in a suitable solution of FVIIa for a period sufficient to ensure that the carrier material is adequately soaked with the FVIIa solution. FVIIa may also be incorporated into the solid carrier by means of spraying, embedding or multiple injections. After vacuum drying or freeze drying to evaporate excess of water the FVIIa impregnated carrier is transferred to a suitable package, such as paper bags or a blister package and terminally sterilized by means of, for instance, heat, ethyleneoxide or ionizing irradiation.

FVIIa may be fixed to the carrier by electrostatic interaction between FVIIa and the carrier material.

FVIIa may also be covalently bound to the carrier by means of chemical crosslinking reagents, such as bifunctional N-hydroxy succinimide esters or other bifunctional chemical crosslinking reagents.

Finally, FVIIa may be fixed to the carrier by physical means such as absorption, dispersion or adsorption.

FVIIa may also be added to the carrier just before use, e.g. by spraying a suitable solution of FVIIa onto the carrier material for by embedding the carrier into a FVIIa solution. Alternatively, the FVIIa solution may be injected into the carrier.

A preferred carrier is a biodegradable sponge material known in the prior art as hemostatic sponges.

Materials for the preparation of hemostatic sponges are conventionally selected from biodegradable or biologically absorbable compounds such as collagen, gelatine, chitin, cellulose, polyglycolic acid and polyacetic acid. Such absorbable hemostats can be left at the site of bleeding even after suturing of internal injuries and will exert their effect over a period of time, dependent on their water solubility, degradability, and size.

The characteristics of the above materials may be conditioned by various chemical or physical treatments resulting in e.g. a preferred improved mechanical strength of the structure or in rendering the material less water soluble thereby retarding the rate of absorption which may extend the period of hemostatic activity.

As an example, gelatine may be denatured by treatment at temperatures in the range of 100-160° C. for several hours. After such treatment the originally water soluble gelatine will become substantially water insoluble but can still be degraded to absorbable molecules by proteolytic enzymes present in the body.

In contrast, hemostatic sponges prepared from undenatured gelatine will dissolve rather rapidly and turn into a soft gel when brought into contact with aqueous solutions or bleeding wounds.

The FVIIa containing dry hemostatic sponge may be prepared either by forming a foam of undenatured gelatine and FVIIa which is thereafter freeze-dried or by saturating a preformed dried sponge with a solution of FVIIa, the wet sponge thereafter being freeze-dried.

The latter technique implies the possibility to apply water insoluble sponge material which may be advantageous because such sponges retain their physical structure after application to the site of bleeding for considerably longer time than undenatured sponges.

In a preferred embodiment of the present invention the carrier is a ready-to-use hemostatic sponge to which FVIIa has been added prior to packaging and terminal sterilization.

### EXAMPLES

### Example 1

Four 5 mm cores of a gelatine sponge (Spongostan® commercially available from Novo Nordisk A/S) were cut using a punch. Two of these were soaked in sterile water and the other two were soaked in two ml of sterile water in which was dissolved 1.13 mg of Factor VIIa. The soaking time was approximately five minutes before application to bleeding sites which were made as described below.

A 450 gram Spraque-Dawley rat was anesthetized with halothane, followed by 0.2 ml/kg of a stock anesthetic solution, which was given intraperitoneally.

The rat was placed on a warming pad and the abdomen was opened with a long, mid-line incision to expose the liver. Gut contents were packed with warm saline swabs.

A piece of steel was placed behind the liver to provide a firm bed. Four 5 mm biopsies were cored through the full thickness of the liver and removed and the four prepared pieces of gelatine sponge were placed into the holes.

These four sites were observed for 20 minutes and at the end of the time the liver was excised and an attempt was made to remove the gelatine plugs by grasping with fine toothed forceps and pulling gently.

The two sites which were plugged with gelatine sponge plugs impregnated with Factor VIIa stopped bleeding, while the two other sites continued to ooze. It was not difficult to remove any of the four plugs from the liver biopsy sites, but it appeared more difficult to remove those soaked in Factor VIIa.

### Example 2

Prior to the surgical intervention, a small piece (45×20×10 mm) was cut out of a dry, gelatine sponge (Spongostan® Standard). The size stated was chosen to ensure that the sponge would exactly cover an incision 25 mm in length with an overlap of 10 mm.

Also an aqueous solution (1.0 mg/ml) of freeze dried Factor VIIa, containing calcium ions (concentration of 10.0 mMol), was made and kept at room temperature prior to the operation.

In an anaesthetized pig, laparotomy was performed through a midline incision and the spleen was delivered into the wound. Incisions were made 3.0 mm deep and 25 mm in length using a special device made from a scalpel mounted with a stop block and a pattern with a linear groove. The first incision was a control incision left for free bleeding for 12 minutes to ensure that coagulation did not occur spontaneously.

Another incision was then made 30 mm apart from the first incision and allowed to bleed freely for 60 seconds. A piece of gelatine sponge was then carefully place upon the incision and 1.0 ml of Factor VIIa solution was dropped onto and gently massaged into the sponge under light finger pressure for 30 seconds. complete hemostasis was obtained momentarily.

The test series did also include four different, commercially available hemostatic sponges moistened with an isotonic Sodium chloride solution. The individual time for hemostasis ranged from 1.8 minutes to 7.5 minutes.

Using the same test procedure bovine thrombin, applied in a watery solution (50 NIH Units/ml), also provoked momentary hemostasis.

### Example 3

Without being incorporated into a matrix, an aqueous solution of Factor VIIa was applied topically as a spray to control venous bleeding from the gallbladder bed and from abdominal surgical incisions. The investigation was divided into two parts involving a total of 8 patients. The study was designed as a double-blind randomized placebo controlled study.

Vials containing 562.5 g of lyophilized Factor VIIa or placebo preparations resembling Factor VIIa were reconstituted with 3.7 ml of sterile water immediately before use and transferred into syringes with sprinkler needles. All 3.7 ml were syringed at each administration.

Four patients undergoing cholecystectomy were investigated, two receiving Factor VIIa and two matching placebo. After removal of the gallbladder, Factor VIIa or placebo was syringed on to the gallbladder bed. Efficacy was assessed 2 minutes later. In each case the efficacy of the preparation was rated comparing ooze before and after application.

Four other patients undergoing general elective abdominal surgery were investigated. Each incision was extended down to but not through the peritoneum with arterial “spurters” being controlled using the surgeon's usual technique. Immediately after the surgical incision the middle of the wound was covered with a thick swab and Factor VIIa was syringed on to the one end of the wound and matching placebo to the other. Efficacy was assessed 3 minutes after the wound was syringed. The surgeon judged blindly which half of the wound was bleeding less.

In these studies, Factor VIIa had no effect in the control of venous bleeding. The likely reason was considered to be that Factor VIIa was washed away from the wound when applied only in an aqueous solution and not incorporated into a matrix or a biologically compatible carrier which would have allowed factor VIIa to remain in contact with the bleeding wound.

The present invention is not to be limited in scope by the above examples since they are intended as single illustrations of the invention. Indeed, various modifications of the invention in addition to those shown and described herein will become apparent to those skilled in the art from the foregoing description. Such modifications are intended to fall within the scope of the appended claims.

1. A method for inducing hemostasis at a bleeding wound comprising providing topically to the site of the bleeding wound a hemostatically effective amount of FVIIa which is unaccompanied by other blood clotting factors and which has sufficient activity alone to produce a hemostatic effect, together with a biologically compatible carrier which permits said factor VIIa to remain in contact with said bleeding wound.

2. A method according to claim 1, wherein the biologically compatible carrier is a gel or a paste.

3. A method according to claim 2, wherein the gel or paste has a viscosity in the range of about 200 cps to about 30,000 cps.

4. A method according to claim 1, wherein the biologically compatible carrier is solid.

5. A method according to claim 1, wherein the biologically compatible carrier is selected from the group consisting of natural macromolecules, chemically modified natural molecules, synthetic polymers, natural or synthetic fibers or mixtures thereof.

6. A method according to claim 1, wherein the biologically compatible carrier is selected from the group consisting of polysaccharides or proteins or mixtures thereof.

7. A method according to claim 4, wherein the biologically compatible carrier is a granule, powder, sponge, film, plaster, surgical dressing or a bandage.

8. A method according to claim 7, wherein the biologically compatible carrier is made of modified cellulose, collagen, gelatine or natural or synthetic fibers.

9. A method according to claim 1, wherein FVIIa is fixed to the biologically compatible carrier by electrostatic interaction between FVIIa and the biologically compatible carrier or by covalent binding of FVIIa to the biologically compatible carrier.

10. A method according to claim 9, wherein FVIIa is bound covalently to the biologically compatible carrier by means of chemical crosslinking reagents, such as bifunctional N-hydroxy succinimide esters or other bifunctional chemical crosslinking reagents.

11. A method according to claim 1, wherein FVIIa is fixed to the biologically compatible carrier by physical means, such as absorption, dispersion or adsorption.

12. A method according to claim 1, wherein the amount of FVIIa is in the range of from about 0.2 to about 2.0 mg.

13. A method according to claim 12, wherein the amount of FVIIa is in the range of from about 0.9 to about 1.1 mg.

14. A hemostatic composition for inducing hemostasis at a bleeding wound comprising a hemostatically effective amount of FVIIa which is unaccompanied by other blood clotting factors and which has sufficient activity alone to produce a hemostatic effect, together with a biologically compatible carrier which permits said factor VIIa to remain in contact with said bleeding wound.

15. Hemostatic composition according to claim 14, wherein the biologically compatible carrier is a gel or a paste.

16. Hemostatic composition according to claim 15, wherein the gel or paste has a viscosity in the range of about 200 cps to about 30,000 cps.

17. Hemostatic composition according to claim 14, wherein the biologically compatible carrier is solid.

18. Hemostatic composition according to claim 14, wherein the biologically compatible carrier is selected from the group consisting of natural macromolecules, chemically modified natural molecules, synthetic polymers, natural or synthetic fibers or mixtures thereof.

19. Hemostatic composition according to claim 18, wherein the biologically compatible carrier is selected from the group consisting of polysaccharides or proteins or mixtures thereof.

20. Hemostatic composition according to claim 17, wherein the biologically compatible carrier is a granule, powder, sponge, film, plaster, surgical dressing or a bandage.

21. Hemostatic composition according to claim 17, wherein the biologically compatible carrier is made of modified cellulose, collagen, gelatine or natural or synthetic fibers.

22. Hemostatic composition according to claim 14, wherein FVIIa is fixed to the biologically compatible carrier by electrostatic interaction between FVIIa and the biologically compatible carrier or by covalent binding of FVIIa to the biologically compatible carrier.

23. Hemostatic composition according to claim 22, wherein FVIIa is bound covalently to the biologically compatible carrier by means of chemical crosslinking reagents, such as bifunctional N-hydroxy succinimide esters or other bifunctional chemical crosslinking reagents.

24. Hemostatic composition according to claim 14, wherein FVIIa is fixed to the biologically compatible carrier by physical means, such as absorption, dispersion or adsorption.

25. Hemostatic composition according to claim 14, wherein the amount of FVIIa is in the range of from about 0.2 to about 2.0 mg.

26. Hemostatic composition according to claim 25, wherein the amount of FVIIa is in the range of from about 0.9 to about 1.1 mg.

27. Hemostatic composition according to claim 14, comprising a fibrinolysis inhibitor such as aprotinin, epsilon-aminocaproic acid or tranexamic acid.

28. Hemostatic composition according to claim 14, further comprising a stabilizer.

29. Hemostatic composition according to claim 28, wherein the stabilizer is selected from the group consisting of naturally occurring amino acids, mono- or disaccharides, polyglycols, glycerol, proteins or divalent metal ions and mixtures thereof.

30. Hemostatic composition according to claim 14, comprising one or more buffering salts selected from alkaline metal acetates, alkaline metal carbonates or hydrogen carbonates, alkaline metal succinates, imidazole, TRIS, and zwitteranionic buffering systems, and mixtures thereof.

31. Hemostatic composition according to claim 14, comprising one or more antimicrobial or bacteriostatic agents selected from antibiotics, sulphonamides, antimycotic agents, antiviral compounds, and preservatives.
